(19) United States
(12) Patent Application Publication
(10) Pub. No.: US 2025/0268779 A1
(43) Pub. Date: Aug. 28, 2025

(54) Finger Massage Apparatus

(71) Applicant: Claudia Ann Ross, Atlanta, GA (US)

(72) Inventor: Claudia Ann Ross, Atlanta, GA (US)

(21) Appl. No.: 19/064,627

(22) Filed: Feb. 26, 2025

Related U.S. Application Data
(60) Provisional application No. 63/558,057, filed on Feb. 26, 2024.

(51) Int. Cl. A61H 15/00; A61M 21/00; A61M 21/02

(52) U.S. Cl. CPC A61H 15/00; A61M 21/02; A61H 2015/0042; A61H 2201/0192; A61H 2201/1253; A61H 2201/1635; A61H 2201/165; A61H 2205/067; A61M 2021/0022

(57) ABSTRACT

A finger massage apparatus for calming and relaxation wherein a bead, wheel, barbell, or other tactile feature within a channel may be used to provide a tactile or massaging sensation to a user. A user may place their finger in the device, or wear the device normally as with jewelry, and then manipulate the bead, wheel, or barbell to massage the finger. The device may be used in conjunction with breathing, focus, or meditation exercises and may be used to help focus the user on those calming activities. The device may include a number of variations, including those based on a ring with a platform, a sleeve, a double-ring structure, or other configurations to provide a variety of wearable devices with adaptable tactile sensations.

### CROSS REFERENCE TO RELATED APPLICATIONS

The present application claims priority under 35 U.S.C. § 119 (e) from U.S. Provisional Application Ser. No. 63/558,057, filed Feb. 26, 2024, entitled “Deep Breathing Fidget Ring,” the disclosure of which is hereby incorporated by reference herein in its entirety.

### FIELD OF THE INVENTION

The present invention relates generally to the field of health and wellness accessories. More specifically, the invention is in the subfield of massage and tactile sensation devices.

### BACKGROUND OF THE INVENTION

Many individuals suffer from anxiety, stress, nervousness, or other mental and physical symptoms of distress. There are a number of methods for addressing these symptoms, including, but not limited to, pharmaceuticals, breathing methods, meditation, and devices such as sound machines or other devices that allow a person to focus or otherwise channel their nervous energy to remain or become calm. However, most of these methods for addressing nervous energy, anxiety, and stress require harsh drugs that require a prescription, or breathing and meditation methods that are difficult to focus on, particularly when an individual may be in a state of mental distress. Furthermore, an individual may not be in a place where it is possible to fully relax or to use a sound machine or other aids to help in focusing and maintaining a breathing or meditation exercise.

As such, there is a need in the art for a simple, effective, and portable device that may help an individual to relax, calm themselves, or participate in methods for reducing stress, anxiety, and nervousness.

### SUMMARY OF THE INVENTION

An aspect of an embodiment of the present invention provides a finger massage apparatus that allows a user to place the device on their finger and then manipulate a bead, wheel, barbell, or other feature to give the user a tactile sensation. The user may manipulate the bead, wheel, barbell, or other feature with another finger on the hand in which the device is mounted, or with their opposing hand. The user may then experience both a direct tactile sensation from the purposeful manipulation of the device and an indirect tactile sensation from the bead, wheel, barbell, or other feature engaging with the user's finger within the finger massage apparatus. The device may be used in conjunction with breathing exercises, with meditation exercises, and to provide a tactile sensation for the user to focus on. The user may also gain some relaxation benefit because the device allows them a small item to play with, manipulate, or fidget. The tactile and massaging function of the present invention also serves to stimulate blood flow, reduce arthritic effects, and may stimulate the vagus nerve, providing additional benefits to the user.

### DESCRIPTION OF THE DRAWINGS

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIGS. 1-3 provide schematic depictions of an exemplary embodiment of a finger massage apparatus.

FIG. 4 provides a schematic depiction of an exemplary embodiment of a finger massage apparatus with a detachable platform.

FIGS. 5 and 6 provide schematic depictions of an exemplary embodiment of a finger massage apparatus with a cuff-like ring.

FIG. 7 provides a schematic depiction of an exemplary embodiment of a finger massage apparatus with a size-adjustable ring.

FIGS. 8 and 9 provide schematic depictions of an exemplary embodiment of a finger massage apparatus with a spherical bead.

FIG. 10 provides a schematic depiction of an exemplary embodiment of a finger massage apparatus with a transverse channel.

FIG. 11 provides a schematic depiction of an exemplary embodiment of a finger massage apparatus with two transverse channels.

FIGS. 12 and 13 provide schematic depictions of an exemplary embodiment of a finger massage apparatus with a sleeve.

FIGS. 14 and 15 provide schematic depictions of an exemplary embodiment of a finger massage apparatus with a sleeve and a sheath for containing a bead.

FIGS. 16-18 provide schematic depictions of exemplary embodiments of a finger massage apparatus with a sleeve in different lengths.

FIG. 19 provides a schematic depiction of an exemplary embodiment of a finger massage apparatus with two channels.

FIGS. 20 and 21 provide schematic depictions of an exemplary embodiment of a finger massage apparatus with two rings.

FIG. 22 provides a schematic depiction of an exemplary embodiment of a clamp style finger massage apparatus.

FIGS. 23A-23F provide schematic depictions of exemplary embodiments of different channel designs for the platform of a finger massage apparatus.

FIGS. 24A-24D provide schematic depictions of exemplary embodiments of platforms with channel detents for a finger massage apparatus.

FIGS. 25A-25F provide schematic depictions of the engagement of a bead, roller, or wheel with the channel of the platform of exemplary embodiments of a finger massage apparatus.

FIGS. 26A-26D provide schematic depictions of exemplary embodiments of beads for a finger massage apparatus.

FIGS. 27A-27D provide schematic depictions of exemplary embodiments of a roller wheel for a finger massage apparatus.

FIG. 28 provides a schematic depiction of an exemplary embodiment of a split bead for a finger massage apparatus.

### DETAILED DESCRIPTION OF EXEMPLARY EMBODIMENTS OF THE INVENTION

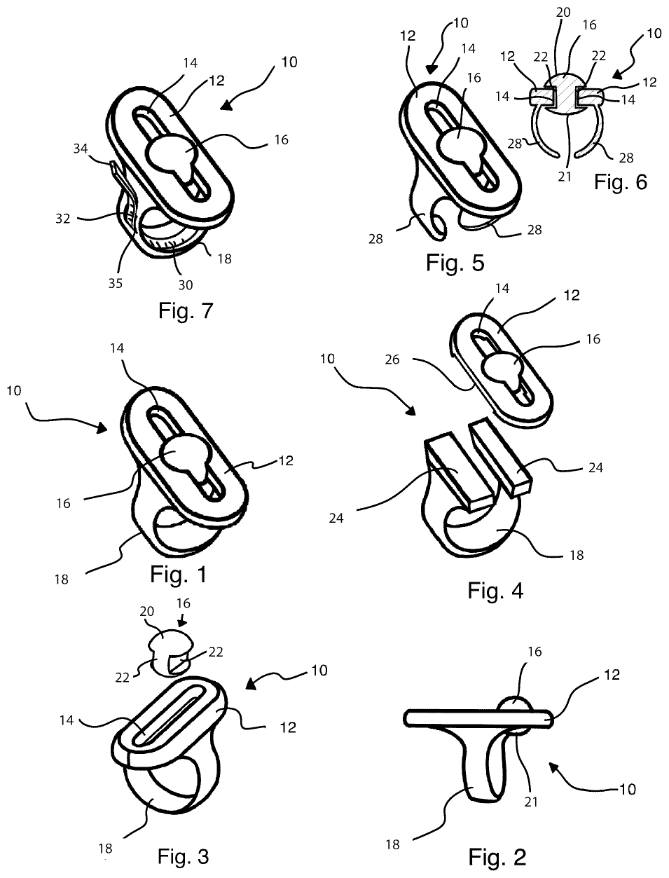

FIGS. 1-3 provide depictions of an exemplary embodiment of a finger massage apparatus 10, which may comprise a platform 12 with a ring 18 affixed or otherwise attached to the bottom surface of the platform 12. The platform 12 may also include or incorporate a channel 14 and a bead 16 disposed within the channel 14. The bead 16 may itself comprise an upper contact surface 20 and a lower contact surface 21, with one or more bead channels 22 disposed between the upper contact surface 20 and the lower contact surface 21. The bead channels 22 are adapted to mate with the channel 14 to allow the bead 16 to translate, slide, or move through the channel 16 when manipulated by a user. It should be appreciated that the ring 18 of the finger massage apparatus 10 may not be completely circular and may incorporate a gap or aperture where the ring 18 contacts the platform 12 of the finger massage apparatus 10 in order to allow the lower contact surface 21 of the bead 16 to translate or move fully through the length of the channel 14.

The upper contact surface 20 of the bead 16 extends above the upper surface of the platform 12. Similarly, the lower contact surface 21 of the bead 16 extends below the lower surface of the platform 12. When a user inserts his or her finger into the ring 18 of the finger massage apparatus 10, the lower contact surface 21 of the bead 16 will be positioned to contact the user's finger while the upper contact surface 20 of the bead 16 will be exposed above the platform 12 such that the user may manipulate the bead 16 and move or displace it through the channel 14. When the user moves or displaces the bead 16 through the channel 14, the user will experience a tactile sensation or massage on the finger which is inserted into the finger massage apparatus 10. Furthermore, the user will experience two types of tactile sensation. Firstly, the user will feel a direct, or active, tactile sensation when the user contacts the upper contact surface 20 of the bead 16 to move or displace the bead 16. Secondly, the user will feel an indirect, or passive, tactile sensation when the lower contact surface 21 of the bead 16 is moved or displaced and contacts the user's finger that is within the ring 18 of the finger massage apparatus 10.

Still referring to FIGS. 1-3, a user may manipulate or use the finger massage apparatus 10 in a number of ways to provide a calming, relaxing, or anxiety-reducing experience. For example, the user may place the finger massage apparatus 10 on their finger with the platform 12 facing upwards as with a traditional ring and use their other hand or fingers to manipulate the bead 16. Similarly, a user may place the platform 12 on the underside, or palm side of their finger and use either a finger from the opposing hand or their thumb to manipulate the bead 16. The user may also use the finger massage apparatus 10 in conjunction with breathing exercises, mantras, meditation, calming music, essential oils, or other calming methods or devices to relax or relieve anxiety. For example, a user may time the manipulation of the bead 16 with their breathing by extending the bead 16 away from them in the channel 14 on inhale and retracting the bead 16 towards them during exhale. However, the user may manipulate the bead 16 and move it through the channel 14 in any way or in any arrangement as desired or required for a particular breathing exercise, meditation exercise, or other calming method. It should be appreciated that the finger massage apparatus 10 may also provide a fidget device to help calm or relax someone with nervous energy or anxiety issues.

FIG. 4 provides an exploded view of a finger massage apparatus 10 with a separable platform 12 and ring 18. The platform 12 of the finger massage apparatus 10 may include or incorporate a channel 14 and a bead 16 disposed with and in slidable communication with the channel 14. The ring 18 may include or comprise one or more ring pads 24. Similarly, the platform 12 may include or comprise a platform recess 26 that is adapted or otherwise configured to mate with the ring pads 24 such that the platform 12 may be removed and reattached to the ring 18. A user may then replace the platform 12 for another platform 12 that may include a differently shaped bead 16 or channel 14 for a different tactile sensation, or for another platform 12 which has a different aesthetic style or design. In this way, a single ring 18 may allow the user to experience multiple physical sensations or to match with different types of outfits, situations, or styles. It should be appreciated that any number of attachment means may be used between the platform 12 and ring 18. For example, the platform 12 may be affixed to the ring 18 via a snap mechanism, a sliding mechanism incorporating dovetails or other geometric features to secure the platform 12 to the ring 18, magnets, fasteners, pins, or any other attachment mechanism that is suitable for a particular application of the finger massage apparatus 10.

FIGS. 5 and 6 provide perspective and cross-sectional views, respectively, of an exemplary embodiment of a finger massage apparatus 10 with cuff horns 28 in place of a ring. As before, the finger massage apparatus 10 may comprise a platform 12 with a channel 14 adapted to accept or otherwise carry a bead 16. The bead 16 may also comprise an upper contact surface 20, lower contact surface 21, and one or more bead channels 22 configured or adapted to mate with or engage the channel 14 of the platform 12. The cuff horns 28 may be made of a flexible, bendable, or springy material such that the finger massage apparatus 10 may be provided with cuff horns 28 sized to fit or grip a relatively small finger while allowing the cuff horns 28 to flex to expand and fit a larger finger without undue discomfort. This expansion and flex of the cuff horns 28 may then allow one finger massage apparatus 10 to comfortably fit a wider variety of finger sizes while providing adequate grip on the user's finger to allow for manipulation of the bead 16 without displacing the finger massage apparatus 10. It should be appreciated that the cuff horns 28, or the ring 18 as shown in other exemplary embodiments of the finger massage apparatus 10, may include an inner lining, material, or texture to provide additional grip of the finger massage apparatus 10 to the user's finger. This additional grip may facilitate manipulation of the bead 16 during breathing or other calming exercises without slipping or displacement of the finger massage apparatus 10.

FIG. 7 provides a schematic depiction of an exemplary embodiment of the finger massage apparatus 10 with a user-adjustable sizing mechanism. The finger massage apparatus may include or comprise a platform 12 with a channel 14, and a bead 16 moveably disposed within the channel 14. A ring 18 may be affixed or otherwise attached to the platform 12 and comprise a sizing aperture 35. The finger massage apparatus 10 may also comprise a sizing band 30 in communication with the underside of the platform 12, the inner perimeter of the ring 18, or both. The sizing band 30, which may be made of a flexible material, may also comprise one or more notches 32 or ridges and a pull tab 34 extending through the sizing aperture 35.

To size the ring 18 of the finger massage apparatus 10, a user may place the ring 18 on their finger with the sizing band 30 in a slack or relaxed position. The user may then pull on the pull tab 34, pulling more length of the sizing band 30 through the sizing aperture 35, decreasing the effective internal circumference of the sizing band 30, and consequently providing a tighter fit to the user's finger. The notches 32 or ridges along the length of the sizing band 30 may then interface or otherwise interact with the edge of the sizing aperture 35 or other features on the ring 18 to provide a ratcheting function wherein the sizing band 30 will maintain tension and sizing until the user releases the sizing band 30 by manipulating the pull tab 34.

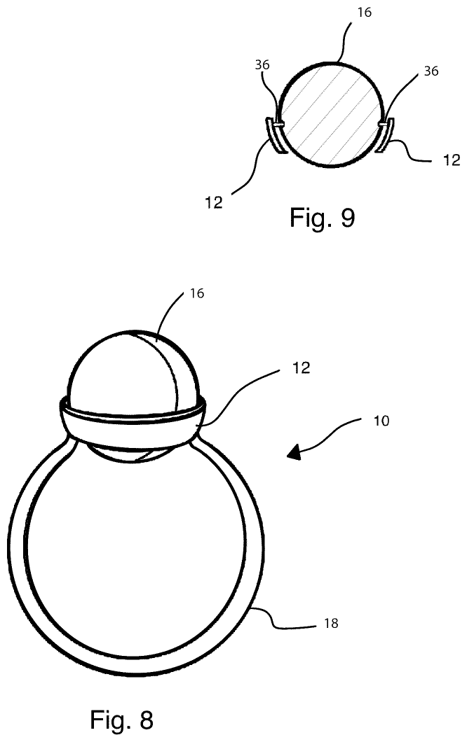

FIGS. 8 and 9 provide a schematic depiction of an exemplary embodiment of a finger massage apparatus 10 and a sectional view of the bead 16 and platform 12, respectively. As shown, the finger massage apparatus 10 may comprise a platform 12 in communication with a ring 18. The platform 12 may comprise a circular structure with a curved cross section to cradle a spherical bead 16. One or more retaining protrusions 36 may extend from the platform 12 to make contact with and interface with the bead 16. It should be appreciated that the retaining protrusions 36 may comprise prongs or other protrusions which grip or interact with the surface of the bead 16, or that the retaining protrusions 36 may interface with a groove or other geometric feature on the bead 16 to retain the bead 16 within the platform 12. Furthermore, it should be appreciated that the spherical bead 16 may roll in one or more directions, allowing for a different tactile sensation for both direct and indirect tactile sensations.

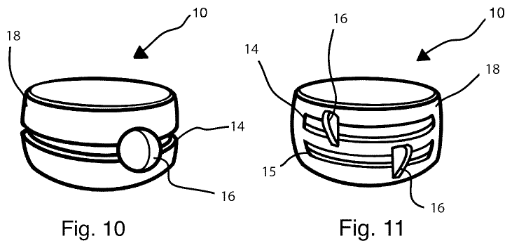

FIGS. 10 and 11 provide depictions of exemplary embodiments of a finger massage apparatus 10 with circumferential channels 14. Referring to FIG. 10, the finger massage apparatus 10 may comprise a ring 18 with a channel 14 and a bead 16 moveably disposed within the channel 14. The channel 14 may be disposed such that the bead 16 moves circumferentially around the ring 18 to provide a different direction of massage compared to a channel 14 which is disposed parallel to, or in a plane parallel to, the central axis of the ring 18 (as shown in FIGS. 1-7). Referring to FIG. 11, the ring 18 may include a first channel 14 and a second channel 15 with a bead 16 disposed within each of the first channel 14 and second channel 15. As shown, both the first channel 14 and second channel 15 may be orientated to guide the bead 16 along the circumference of the ring 18 to provide a different tactile sensation to the user. In certain embodiments, as shown, the bead 16 may take on a different shape to provide a different tactile sensation. For example, the bead may be flat with a curved upper surface, may have a spherical or hemispherical upper surface, or may have an upper surface that is pointed, flattened, or in any shape as desired or required for a particular application.

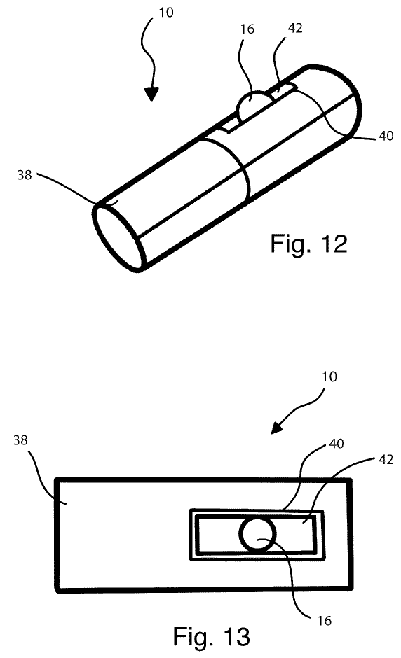

FIGS. 12 and 13 provide depictions of exemplary embodiments of a finger massage apparatus 10 having a sleeve 38 for enclosing or attaching to a user's finger. The sleeve 38 may be in communication with a frame 40, which may enclose a frame channel 42. A bead 16 may be disposed within the frame channel 42 and may slide, roll, or both, along the length of the frame channel 42. It should be appreciated that the sleeve 38 may be made of a rigid or flexible material. In the case of the flexible material, the sleeve 38 may be sized such that the sleeve 38 expands to fit snugly around the user's finger while allowing the user to flex or move their finger freely. In certain embodiments, the frame 40 may be molded directly into the material of the sleeve 38, or the frame 40 may be a separate piece that is affixed or otherwise attached to the sleeve 38 through any means including, but not limited to, adhesives, fasteners, friction welding, or any other attachment means as desired or required for a particular application. Furthermore, it should be appreciated that the bead 16 may extend above the outer surface of the sleeve 38 and below the inner surface of the sleeve 38 such that a user may manipulate the bead 16 while it is in contact with the user's finger in the sleeve 38.

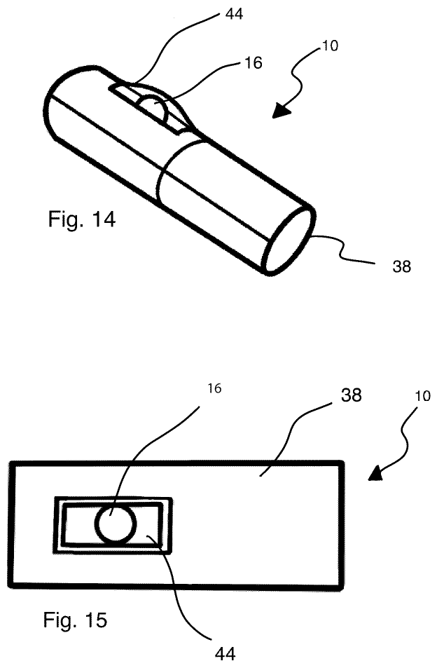

FIGS. 14 and 15 provide depictions of an exemplary embodiment of a finger massage apparatus 10 with a sleeve 38. A bead 16 may be held in place by a sheath 44 of flexible material. The sheath 44 may be shaped or otherwise configured into an elongated or other shape to allow the bead 16 to move within the sheath 44 and provide a massaging function similar to embodiments of the finger massage apparatus 10 described with reference to other figures herein. For example, the sheath 44 may be rectangular or oval in shape, and may be a covering molded into, adhesively attached, or otherwise attached to a sleeve 38 made of a flexible material to entrain the bead 16 within the space between the sleeve 38 and sheath 44. The bead 16 may then slide fore and aft within the space between the sleeve 38 and sheath 44 to provide a massaging function. In certain embodiments, the sleeve 38 may be made of either a flexible or rigid material and may contain an aperture that is covered both on the inner surface of the sleeve 38 and the outer surface of the sleeve 38 by the sheath 44. In such embodiments, the sheath 44 may act like a bag or enclosure to entrain the bead 16 and allow the bead 16 to be moved within the space enclosed by the sheath 44 to provide a massaging function.

It should be appreciated that the finger massage apparatus 10 may include or comprise any number of materials or features. For example, the bead 16 may take on any number of shapes, sizes, or geometries, and the sleeve 38 may comprise any materials, including both rigid and flexible materials. Furthermore, the sleeve 38 may include materials of different colors, transparent or opaque materials, or may serve as the background or substrate for designs or other visual flourishes.

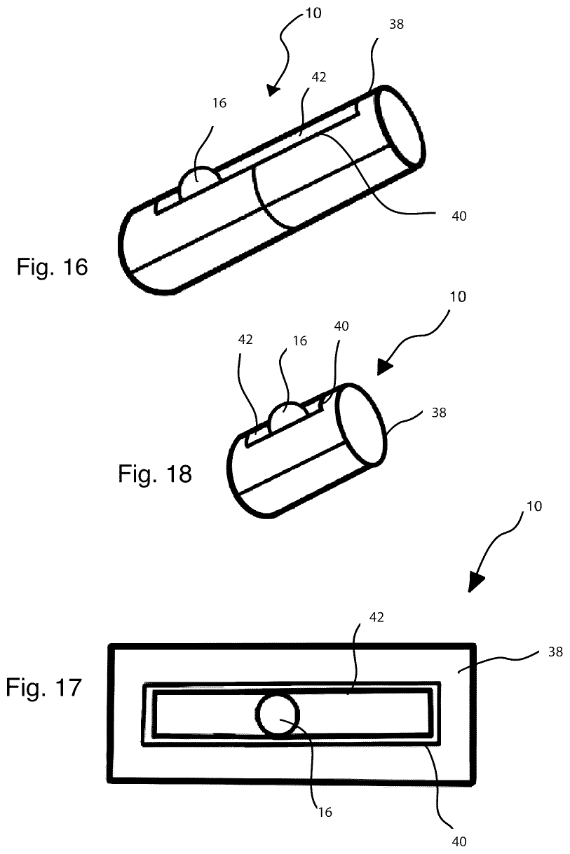

FIGS. 16-18 provide depictions of exemplary embodiments of a finger massage apparatus 10 with a sleeve 38. The finger massage apparatus 10 may include any number of variations or alterations to its geometry and features to be adapted for a particular use or application. For example, referring to FIGS. 16 and 17, the sleeve 38 may be longer such as to enclose the user's finger across more than one knuckle. In certain embodiments, the sleeve 38 may be long enough to encompass the proximal and medial phalanges, or the proximal, medial, and distal phalanges. Similarly, the finger massage apparatus 10 may include a frame 40 and frame channel 42 that extends across the entire length or any portion of the length of the sleeve 38. As shown, the frame 40, and consequently the frame channel 42, may extend across the entire length of the sleeve 38 to allow the bead 16 to move along the entire length of the finger.

Referring to FIG. 18, the finger massage apparatus 10 may comprise a sleeve 38 that is shorter and intended to only cover one of the proximal, medial, or distal phalanges. The shorter sleeve 38 may then be in communication with a shorter frame 40, and a shorter frame channel 42. This shorter frame 40 and frame channel 42 provides a shorter path for the movement or translation of the bead 16 to provide a more focused massage experience.

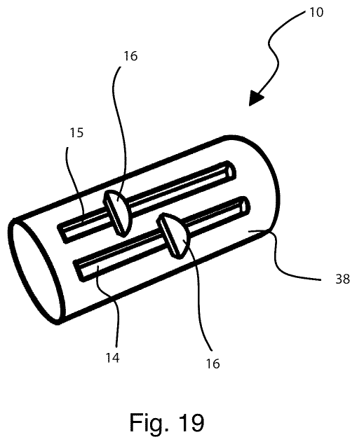

FIG. 19 provides a depiction of yet another exemplary embodiment of a finger massage apparatus 10 with a sleeve 38. The sleeve 38 may comprise a first channel 14 and a second channel 15, each respectively in moveable communication with a bead 16. As shown, the bead 16 in the first channel 14 and the bead 16 in the second channel 15 may move independently from one another offering a user a variety of massage or calming options such as movement of the beads 16 in unison, movement of the beads 16 in opposite directions, or any combination thereof.

Referring to FIGS. 12-19, the finger massage apparatus 10 with a sleeve 38 may be integrated into one or more fingers of a glove, mitten, or other hand-wearable accessories or clothing.

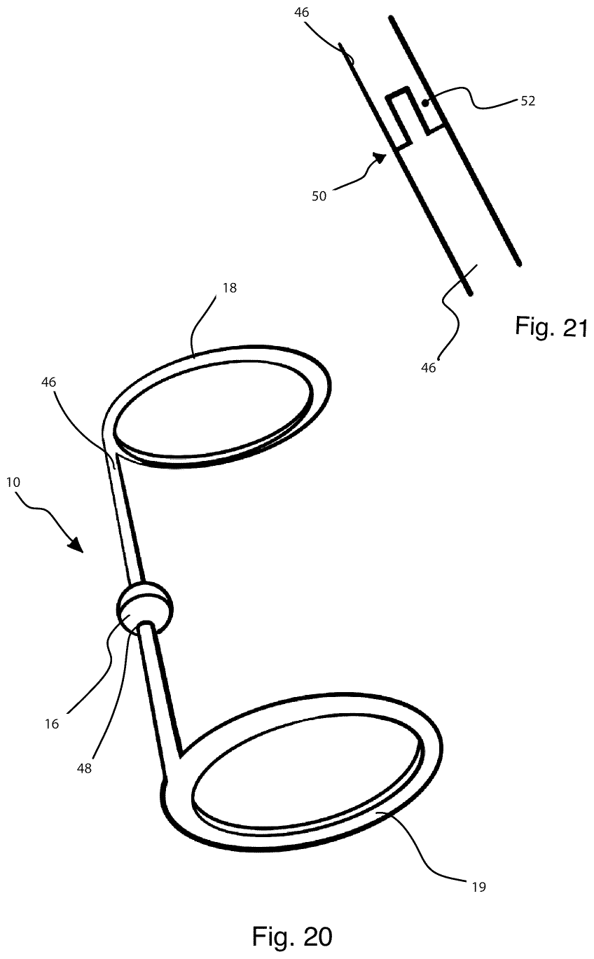

FIGS. 20 and 21 provide depictions of exemplary embodiments of a two-ring finger massage apparatus 10 and an exemplary embodiment of a joint. The finger massage apparatus 10 may comprise a first ring 18 and a second ring 19 in communication with the ends of a connecting bar 46 disposed between the first ring 18 and second ring 19. A bead 16, comprising a bead channel 48, may then be in slidable or moveable communication with the connecting bar 46 as the bead 16 may ride along the connecting bar 46 as it passes through the bead channel 48.

It should be appreciated that the connecting bar 46 may comprise a flexible or rigid material. Furthermore, the first ring 18, second ring 19, and connecting bar 46 may comprise a single molded piece or multiple pieces of the same or dissimilar materials joined together. In certain embodiments, the bead 16 may take on any shape or geometry as desired or required, or as described with reference to other figures herein. Similarly, the connection bar 46 may be straight, curved, or any other shape or geometry as desired or required or as described with reference to other figures herein.

Referring specifically to FIG. 21, the connection bar 46 may comprise a joint 50 or other mechanism for allowing flex, displacement, or movement along the length of the connection bar 46. As shown, the joint 50 may be secured by a pin, rivet, or axle 52 that allows the joint 50 to pivot or rotate about the axle 52. It should be appreciated that the joint 50 may be any type of joint as is necessary or as desired or required including, but not limited to ball joints, slip joints, leaf joints, or any other type of joint that allows pivoting or rotational movement.

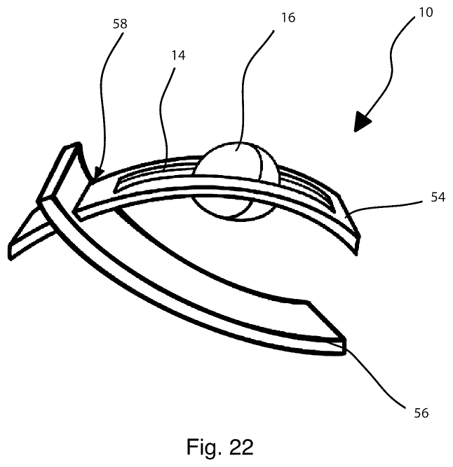

FIG. 22 provides a depiction of an exemplary embodiment of a clamp style finger massage apparatus 10. The finger massage apparatus 10 may comprise an upper flange 54 in communication with a lower flange 56 via a joint 58. A channel 14 in the upper flange 54 may be in moveable communication with a bead 16, which may slide, rotate, or otherwise translate through the channel 14 to massage a user's finger.

Still referring to FIG. 22, the clamp style finger massage apparatus 10 may be adapted for use on the finger tip of a user's finger, or be used across the user's finger at any point along the finger's length. Furthermore, in certain embodiments, the joint 58 may be a solid joint wherein the upper flange 54 and lower flange 56 flex to accept a user's finger. In still further embodiments, the joint 58 may be a hinge, pinned joint, riveted joint, or any other mechanical joint allowing the upper flange 54 and the lower flange 56 to pivot, rotate, or otherwise increase or decrease the spacing between the upper flange 54 and the lower flange 56.

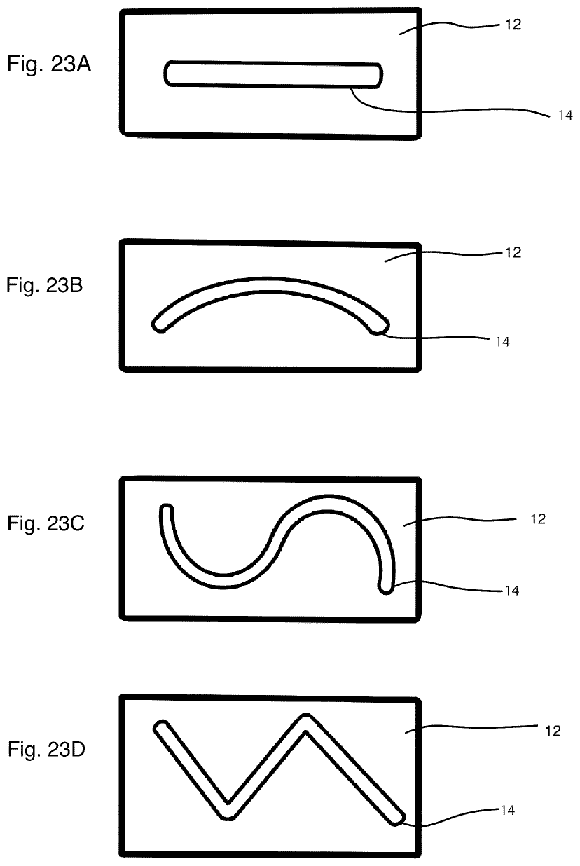

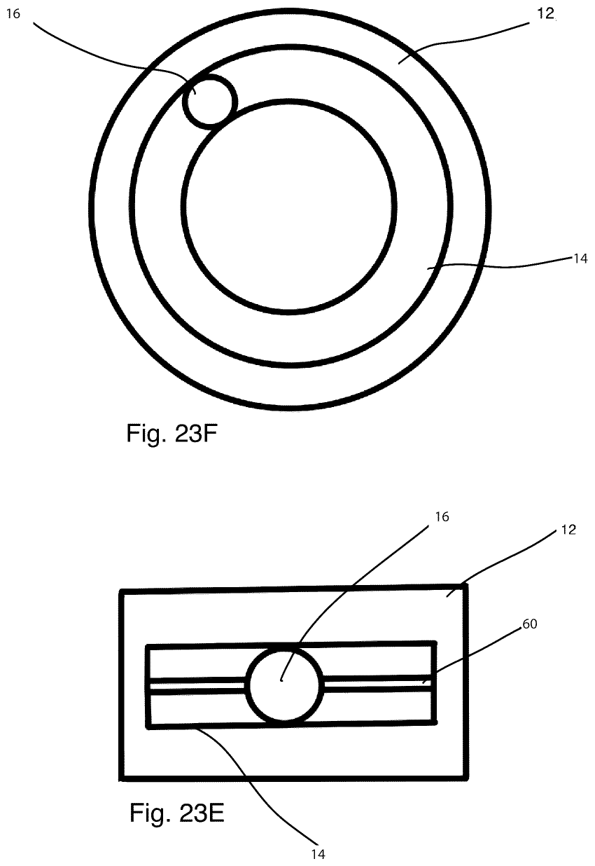

FIGS. 23A-23F provide depictions of several exemplary embodiments of different channel 14 shapes or configurations in a platform 12. It should be appreciated that any of the shown channel 14 shapes, or any other configuration of channel 14, may be used in the platform 12, sleeve 38, or upper flange 54 of the finger massage apparatuses 10 described herein. For example, as shown in FIG. 23A, the channel 14 within the platform 12 may be of a straight configuration. The channel 14 may also be a single curve or C-curve as shown in FIG. 23B. In certain embodiments, the channel 14 may take on an S-curve shape, or a shape with at least one inflection point between concave and convex, as shown in FIG. 23C. Similarly, the channel 14 may take on a zig-zag shape, or a shape wherein the direction of motion of a bead 16 (not shown) would have abrupt changes of direction as illustrated in FIG. 23D. It should be appreciated that the various geometries of the channel 14 as displayed in FIGS. 23A-23D may be adapted for use with any style of bead 16 and may be orientated either parallel with the bones of the user's finger or perpendicular to the bones of the user's finger. Furthermore, the use of different channel 14 geometries allows a user to experience different sensations or different types of tactile movement which may be adapted for a particular breathing exercise or other calming methods.

Referring to FIG. 23E, the channel 14, which is disposed within the platform 12, but may also be used in conjunction with a sleeve 38 or an upper flange 54 of a finger massage apparatus 10, may comprise a channel bar 60 and a bead 16 which rides along the channel bar 60. The channel bar 60 may facilitate different geometries of bead 16, and allow the bead 16 to both translate along the channel bar 60 and rotate about the channel bar 60 during use.

Referring specifically to FIG. 23F, the platform 12, or sleeve 38 or upper flange 54, of a finger massage apparatus, may have a circular or substantially circular channel 14 and a bead 16 within the circular channel 14. It should be appreciated that other closed-loop shapes of channel 14 may be used. For example, the channel 14 may be oval, elliptical, square, triangular, or any other shape as desired or required for a particular application.

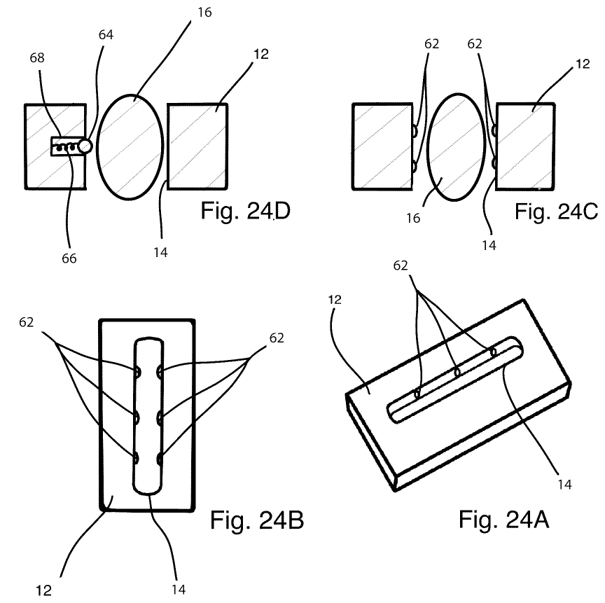

FIGS. 24A-24C provide depictions of exemplary embodiments of a platform 12 and channel 14 with detents or other mechanisms for providing intermittent resistance to a bead 16 as it travels along the length of the channel 14. As shown in FIGS. 24A-24C, the channel 14 may include or comprise one or more obstructions 62, herein illustrated as nubs 62, that may provide additional resistance to a bead 16 at certain specific locations along the length of the channel 14. These locations of additional resistance allow a user to push past the resistance provided by the nubs 62 and give an additional tactile sensation of pushing through the resistance or a “click” feeling to the user during use. It should be appreciated that the nubs 62 may be molded directly into the material of the platform 12, or they may be molded over of a different material, or may be attached or otherwise affixed to the platform 12.

Referring to FIG. 24D, the platform 12 may also incorporate a detent channel 68 within the channel 14 and a ball 64 with a spring 66 to provide the detent function to the finger massage apparatus. When a bead 16 moves along the channel 14 it may encounter the ball 64 and experience resistance to further motion along the channel 14. When the user applies an additional amount of force, the bead 16 will push the ball 64 against the spring 66, retracting the ball 64 into the detent channel 68. When the ball 64 retracts into the detent channel 68, it will clear the channel 14 and allow the bead 16 to pass.

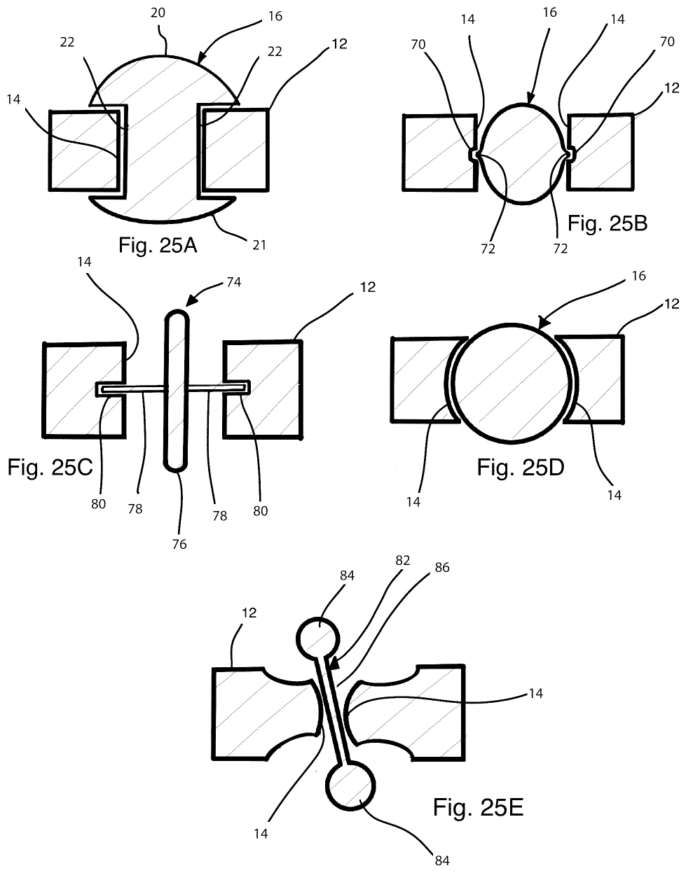

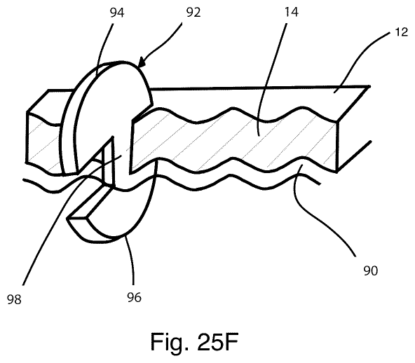

FIGS. 25A-25F provide depictions of exemplary embodiments of the engagement of a bead 16, wheel 74, barbell 82, or slider 92 with the channel 14 of a platform 12. Referring to FIG. 25A, the bead 16 may comprise an upper contact surface 20, a lower contact surface 21, and one or more bead channels 22. The one or more bead channels 22 may be shaped, configured, or otherwise adapted to engaged with or be complementary to the shape of the channel 14. The engagement of the bead channels 22 with the channel 14 allows the bead 16 to translate or slide along the channel 14 while not being able to escape the channel 14. In another exemplary embodiment, as shown in FIG. 25B, the bead 16 may comprise one or more bead protrusions 72 which are configured or otherwise adapted to engage one or more channel tracks 70, which may be located within the channel 14. It should be appreciated that the bead protrusions 72 may allow the bead to both slide along the channel 14 while also rotating. In certain embodiments, a spherical or substantially spherical bead 16 may engage the channel tracks 70 without the need for a bead protrusion 72 while still constraining the motion of the bead 16 such that it does not fall out of the channel 14.

Referring now to FIG. 25C, the finger massage apparatus 10 may include a wheel 74 within the channel 14 in place of the bead 16. The wheel 74 may itself comprise a disk 76 in communication with an axle 78. The axle 78 may interface with an axle channel 80 within the channel 14. The wheel 74 may then translate along the channel 14 and rotate about the axle 78.

Referring to FIG. 25D, the channel 14 may have a curved shape that allows a spherical, or substantially spherical, bead 16 to be entrained within the channel 14. The bead 16 may then rotate about any axis and translate or roll along the length of the channel 14.

Referring to FIG. 25E, the finger massage apparatus 10 may include a barbell 82 within the channel 14. The barbell 82, which may comprise one or more balls 84 in communication with a shaft 86, may pass through the channel 14 such that the balls 84 may extend beyond the surface of the platform 12. A user may then manipulate the barbell 82 in a similar fashion to a lever in which the lower ball 84 will move in a motion that is opposite to the upper ball 84 as the shaft pivots about in the channel 14. It should be appreciated that the channel 14 may be an elongated channel to allow the barbell 82 to pivot and translate along the length of the channel 14. However, in certain embodiments, the channel 14 may be shortened or otherwise constricted such that the barbell 82 may pivot fore and aft or side to side, or pivot in a circular motion but not translate along a length of the platform 12. As shown, the platform 12 may include additional depressions or clearances on the top surface, bottom surface, or both, to allow a wider range of motion of the barbell 82.

Referring to FIG. 25F, the finger massage apparatus 10 may also replace the bead 16 with a slider 92. The slider 92, which may comprise an upper portion 94 in communication with a lower portion 96 via a connector 98. The slider 92 may have a more planar type of geometry, which provides a user with a different tactile sensation during use.

Still referring to FIG. 25F, the channel 14 may comprise or include a channel track 90 which provides an additional dimension of movement to the slider 92 as it passes along the channel 14. As shown, the channel track 90 may have a geometry that is not flat or straight, but rather comprises a wave, bumps, or other geometry that tends to move the slider 92 in a vertical direction as it translates through the channel 14. The vertical movement of the slider 92, or a bead 16, wheel 74, or barbell 82 as illustrated with respect to other embodiments of the finger massage apparatus 10, may provide changes to pressure to the user's finger to enhance the massaging or calming effects of the finger massage apparatus 10. It should be appreciated that the connector 98 may take on a geometry to allow the connector 98 to ride along the channel track 90 while permitting the upper portion 94 and lower portion 96 of the slider 92 to extend above and below the surface of the platform 12, respectively. In certain embodiments, the connector 98 may have a wedged or tapered shape to allow the taper to ride along the channel track 90 while the narrower portion of the connector passes through the channel 14. Similarly, the connector 98 may comprise a wider section with shoulders that ride along the channel track 90 and a narrower section that may pass completely through the channel 14. However, any geometry of the connector 98 that allows for engagement of a channel track 90 while passing through the channel 14 may be used.

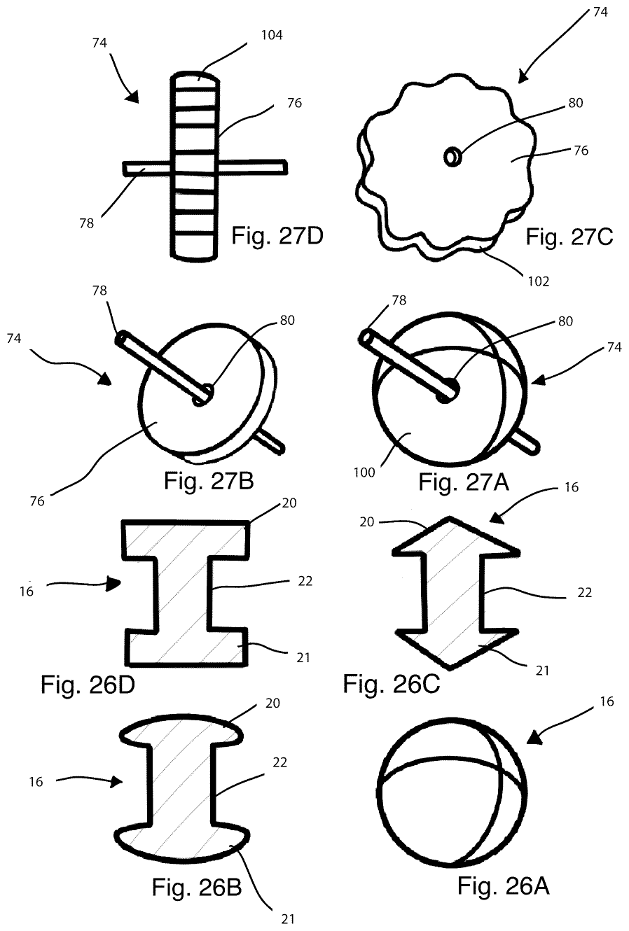

FIGS. 26A-26D provide depictions of various exemplary embodiments of a bead 16. The bead 16 may comprise a spherical or substantially spherical shape as shown in FIG. 26A. The bead 16 may also comprise a shape with a rounded or hemispherical upper contact surface 20 and lower contact surface 21 and one or more bead channels 22, as shown in FIG. 26B. Referring to FIG. 26C, the bead 16, which may comprise one or more bead channels 22, may also comprise a pointed, triangular, or pyramidal shape to the upper contact surface 20, lower contact surface 21, or both. FIG. 26D depicts a bead 16 with a flat, or squared upper contact surface 20 and lower contact surface 21 with one or more bead channels 22 disposed therebetween.

Still referring to FIGS. 26A-D, it should be appreciated that the different shapes of the upper contact surface 20 and lower contact surface 21 may provide a user with different tactile sensations. For example, a rounded contact surface may provide a smoother tactile pressure than a pointed, or pyramidal contact surface, which will provide a more localized pressure to the user. A flat contact surface may provide the most evenly distributed tactile sensation to the user. In certain embodiments, the bead 16 may include an upper contact surface 20 of one configuration with a lower contact surface 21 of a different configuration. In still further embodiments, a bead 16 may be spherical or may have an ellipsoidal or otherwise elongated geometry such that the bead 16 may provide differing amounts of pressure to a user's finger as the bead 16 rotates and engages with the user's finger to a greater or lesser degree.

FIGS. 27A-27D provide depictions of exemplary embodiments of a wheel 74 for use in a finger massage apparatus 10. A wheel 74 may comprise an axle channel 80, which may receive or otherwise interface with an axle 78. The wheel 74 may then include a sphere 100, which may also include an axle channel 80, as shown in FIG. 27A. In certain embodiments, the wheel 74 may comprise a disc 76, as shown in FIG. 27B. Referring to FIG. 27C, the disc 76 may comprise one or more disc protrusions 102 disposed about the circumference of the disk 76 to provide additional tactile sensations to a user of the finger massage apparatus 10. Now referring to FIG. 27D, the disc 76 of the wheel 74 may include a texture 104 or other geometric features on or about the circumference of the disc 76.

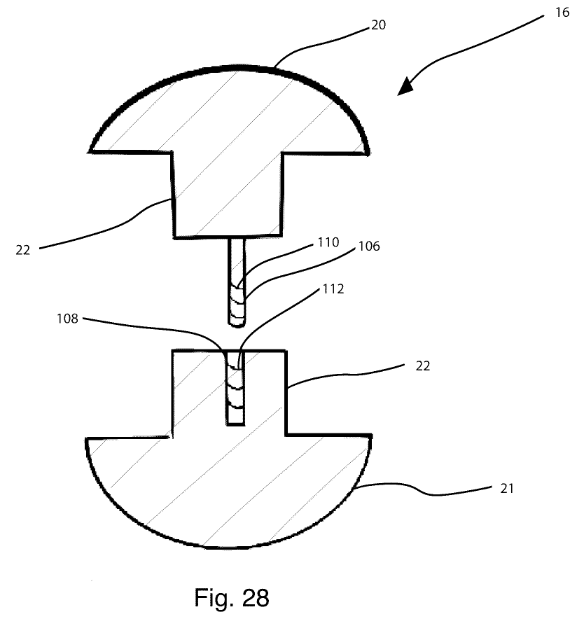

FIG. 28 provides a depiction of an exemplary embodiment of a bead 16 for use in a finger massage apparatus 10 which may be split or otherwise separable to facilitate the interchangeability of multiple beads 16 with a single finger massage apparatus 10. The bead 16, which may comprise an upper contact surface 20, a lower contact surface 21, and one or more bead channels 22, may further comprise a bead pin 106 in communication with a first half of the bead 16 and a pin recess 108 in communication with a second half of the bead 16. The bead pin 106 may comprise one or more bead pin ridges 110 or other geometric features. Similarly, the pin recess 108 may comprise complementary ridges 112 or other geometric features. The bead 16 may then be pulled apart to separate the bead 16 into two separate halves. The bead 16 halves may then be extracted from the channel 14 of a finger massage apparatus 10. A user may then re-connect the two halves of the bead 16 by engaging the bead pin 106 with the pin recess 108. The bead pin ridges 110 and pin recess ridges 112 allow for a stronger, or more secure connection between the two halves of the bead 16.

Still referring to FIG. 28, it should be appreciated that any number of variations of connection mechanisms may be used to secure the halves of the bead 16 together. For example, the two halves of a bead 16 may be secured together using fasteners, magnets, or other connection means as desired or required for a particular application.

Referring to FIGS. 25E and 26A-27D, it should be appreciated that the bead 16, wheel 74, and barbell 82 may take on any number of configurations, geometries, or features when used in the finger massage apparatus 10. For example, the bead 16, wheel 74, and barbell 82 may be made of any number of materials including polymers, elastomers, metals, glass, or any other suitable material. Furthermore, the bead 16, wheel 74, and barbell 82 may include or incorporate decorative materials for aesthetic appearance. In certain embodiments, the bead 16, wheel 74, and barbell 82 may include stones, including gemstones, crystals, or the like which may provide a pleasing appearance or which may be perceived by a user to enhance the relaxation or healing aspects of the finger massage apparatus. It should also be appreciated that any shape, geometry, or feature discussed in reference to a bead 16, wheel 74, or barbell 82 may be adapted for use on another embodiment of a bead 16, wheel 74, or barbell 82. In still further embodiments, the bead 16, wheel 74, or barbell 82 may include or comprise different textures or material selections to provide a particular tactile experience. For example, the bead 16, wheel 74, or barbell 82 may include knurling, stippling, or other roughening textures, or may include specific metals or plastics to provide more or less heat transfer to the user's finger.

Referring to FIGS. 1-25E, it should be appreciated that any features, geometries, or other aspects of the finger massage apparatus 10 described with reference to any one exemplary embodiment may be applied to another exemplary embodiment of the finger massage apparatus 10. Furthermore, the different geometries and shapes of the channel 14 may be applied to any embodiment, and the connecting bar 46 may also take on a variety of shapes and geometries to provide different paths of travel for the bead 26. It should be appreciated that any element, part, section, subsection, or component described with reference to any specific embodiment above may be incorporated with, integrated into, or otherwise adapted for use with any other embodiment described herein unless specifically noted otherwise or if it should render the embodiment device non-functional. Furthermore, multiple embodiment devices may be combined, incorporated, or otherwise integrated into one another to construct or develop further embodiments of the invention described herein.

In summary, while the present invention has been described with respect to specific embodiments, many modifications, variations, alterations, substitutions, and equivalents will be apparent to those skilled in the art. The present invention is not to be limited in scope by any of the specific embodiments described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device non-functional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments of the invention described herein.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particular interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

I claim:

1. A finger massage apparatus comprising:
a platform comprising an upper surface and a lower surface;
a channel;
a bead; and
a ring in communication with said lower surface of said platform;
wherein said channel is disposed within said platform and said bead is in moveable communication with said channel, and wherein a first portion of said bead extends above said upper surface of said platform and a second portion of said bead extends below said lower surface of said platform.

2. The finger massage apparatus of claim 1, wherein said bead further comprises at least one bead channel;
said bead channel adapted to engage said channel of said platform.

3. The finger massage apparatus of claim 1, wherein said bead is removeable and replaceable with at least one second bead.

4. The finger massage apparatus of claim 3, wherein said bead comprises an upper portion and a lower portion; and
a bead pin in communication with said upper portion of said bead and a pin recess in communication with said lower portion of said bead;
wherein said bead pin may engage said pin recess to secure said upper portion of said bead to said lower portion of said bead.

5. The finger massage apparatus of claim 1, wherein said platform is removably affixed to said ring.

6. The finger massage apparatus of claim 1, wherein said ring comprises a first horn and a second horn;
wherein said first horn in is communication with said lower surface of said platform and said second horn is in communication with said lower surface of said platform.

7. The finger massage apparatus of claim 1, wherein said channel comprises a path shape selected from the group consisting of straight, C-curve, S-curve, and zig-zag.

8. The finger massage apparatus of claim 1, wherein at least one of said first portion of said bead and said second portion of said bead comprises a shape selected from the group consisting of spherical, hemispherical, flat, pyramidal, and rounded.

9. The finger massage apparatus of claim 1, further comprising a second channel and a second bead;
wherein said second channel is disposed within said platform and said second bead is in moveable communication with said second channel, and wherein a first portion of said second bead extends above said upper surface of said platform and a second portion of said second bead extends below said lower surface of said platform.

10. The finger massage apparatus of claim 1, further comprising a sizing band disposed within said ring; and
said sizing band comprising one or more notches and a pull tab;
wherein said pull tab of said sizing band may extend through a sizing aperture in said ring to decrease the effective diameter of said ring and adapt said ring to a smaller finger.

11. The finger massage apparatus of claim 1, further comprising one or more detents;
said one or more detents disposed within said channel and interfering with said bead to generate a holding force when said bead engages with said one or more detents.

12. A finger massage apparatus comprising:
a cylindrical sleeve having an outer surface and an inner surface; and
a bead comprising a first portion of said bead and a second portion of said bead in communication with said cylindrical sleeve;
wherein said bead is in moveable communication with said cylindrical sleeve and wherein said first portion of said bead extends beyond said outer surface of said cylindrical sleeve and said second portion of said bead extends beyond said inner surface of said cylindrical sleeve.

13. The finger massage apparatus of claim 12, wherein said cylindrical sleeve comprises a flexible material.

14. The finger massage apparatus of claim 12, further comprising a frame, said frame enclosing a channel;
wherein said frame is in communication with said cylindrical sleeve, and said bead is in moveable communication with said channel.

15. The finger massage apparatus of claim 12, further comprising a sheath;
wherein said sheath is in communication with said cylindrical sleeve and encloses a space; and
wherein said bead is moveably disposed within said space defined by said sleeve.

16. The finger massage apparatus of claim 12, further comprising a channel in communication with said cylindrical sleeve;
wherein said channel is disposed along an axis of said cylindrical sleeve.

17. The finger massage apparatus of claim 12, further comprising a channel in communication with said cylindrical sleeve;
wherein said channel is disposed along a circumference of said cylindrical sleeve.

18. A finger massage apparatus comprising:
a first ring;
a second ring;
a connection bar having a first end and a second end; and
a bead;
wherein said first ring is in communication with said first end of said connection bar, said second ring is in communication with said second end of said connection bar, and said bead is in slidable communication with said connection bar and disposed between said first ring and said second ring.

19. The finger massage apparatus of claim 18, wherein said connection bar comprises a flexible material.

20. The finger massage apparatus of claim 18, wherein said connection bar comprises a first portion and a second portion; and
wherein said first portion of said connection bar is pivotably affixed to said second portion of said connection bar through a joint.
